(19) United States
(12) Patent Application Publication
(10) Pub. No.: US 2003/0163147 A1
(43) Pub. Date: Aug. 28, 2003

(54) Apparatus and method for using a vascular introducer with an ultrasonic probe

(76) Inventors: Robert A. Rabiner, North Reading, MA (US); Bradley A. Hare, Chelmsford, MA (US); James H. Loper, Wales, MA (US)

Correspondence Address: PALMER & DODGE, LLP, RICHARD B. SMITH, 111 HUNTINGTON AVENUE, BOSTON, MA, 02199, US

(21) Appl. No.: 10/080,787

(22) Filed: Feb. 22, 2002

(51) Int. Cl. A61B 17/22

(52) U.S. Cl. 606/159

(57) ABSTRACT

An apparatus and a method for using a vascular introducer with an ultrasonic probe to remove a debris from a graft, fistula, vessel, port, or other vascular access device. The vascular introducer for insertion into a vascular access device includes an elongated shaft having a distal end and a proximal insertion end; an anchoring mechanism that resides within the elongated shaft when the anchoring mechanism is in a retracted position and extends beyond the proximal insertion end of the elongated shaft and engages an inner surface of the vascular access device when the anchoring mechanism is in an extended position; and an activation mechanism that moves the anchoring mechanism from the retracted position to the extended position. The vascular introducer may further include a rotation mechanism that allows the vascular introducer to change direction within the vascular access device without being removed from the vascular access device.

### RELATED APPLICATIONS

None.

### FIELD OF THE INVENTION

The present invention relates to a vascular introducer to be used in mammals, and more specifically to a vascular introducer used in conjunction with an ultrasonic probe, or similar instrument, to remove debris from a graft, fistula, vessel, port, or other device providing vascular access in a patient with minimal invasiveness and minimal risk to the patient.

### BACKGROUND OF THE INVENTION

Healthy humans have two kidneys, each about the size of an adult fist, located on either side of the spine just below the rib cage. Although the kidneys are small, the kidneys perform many complex and vital functions that keep the rest of the body in balance. For example, kidneys help remove waste and excess fluid, filter the blood (keeping some compounds while removing others), control the production of red blood cells, release hormones that help regulate blood pressure, make vitamins that control growth, and help regulate blood pressure, red blood cells, and the amount of certain nutrients in the body, such as calcium and potassium.

Dialysis is a process of removing waste products and excess fluid which build up in the body when the kidneys are not functioning effectively. The word “dialysis” comes from the Greek “dia”—to pass through, and “leuin” meaning to loosen. Dialysis is necessary when a patient's kidneys can no longer take care of the patient's bodily needs. Dialysis is a medical procedure routinely used in end-stage renal disease (ESRD), also known as end stage kidney failure, usually by the time the patient has lost about 85 to 90 percent of kidney function. Dialysis, as a regular treatment, began in 1960 and is now a standard treatment all around the world. Thousands of patients have been helped by dialysis treatment.

Like healthy kidneys, dialysis keeps the patient's body in balance by removing waste, salt and extra water to prevent them from building up in the body, keeping a safe level of certain chemicals in the patient's blood, such as potassium, sodium and bicarbonate, and helping to control blood pressure. Dialysis uses a membrane as a filter and a solution called dialysate to regulate the balance of fluid, salts and minerals carried in the bloodstream. The membrane may be man-made as in hemodialysis or natural as in peritoneal dialysis.

Hemodialysis is a medical procedure used routinely in the treatment of end-stage renal disease, in which the patient's blood is shunted from the body through a hemodialyser for diffusion and ultrafiltration, and then returned to the patient's vascular system. Hemodialysis removes certain elements from the blood by virtue of the difference in the rates of their diffusion through a semipermeable membrane, for example, by means of a hemodialysis machine or a filter. In hemodialysis, a hemodialyser (commonly referred to as an artificial kidney) is used to clean a patient's blood by removing waste and extra chemicals and fluid from the patient's blood. A hemodialyser works on the principle of blood flowing along one side of a semi-permeable cellulose membrane or a similar product, while the dialysate flows along the other side. The dialysate contains a regulated amount of minerals normally present in the blood, but in renal failure they are present in excess. The membrane has tiny holes of different sizes so that the excess fluid and substances in the blood pass through at different rates, small molecules quickly and larger ones more slowly, to be taken away in the dialysate until a correct balance in the blood is achieved.

During hemodialysis, a kidney machine regulates blood flow, pressure and the rate of exchange. As only a very small amount of blood is in the hemodialyser at any given time, blood needs to circulate from patient to hemodialyser and back to patient for approximately four hours. Hemodialysis treatments typically occur three times per week, with the time and strength of hemodialysis programmed for each patient.

To get the patient's blood into the hemodialyser, there must be an access (entrance) into the patient's blood vessels. A hemodialysis access, or a vascular access, is a way to reach the blood for hemodialysis. For hemodialysis, the following three types of vascular access are predominant: (1) an AV (arterivenous) fistula; (2) an AV (arterivenous) graft; and (3) a catheter. Such access is usually accomplished by minor surgery to a patient.

AV fistulas are formed internally by a surgical anastomosis joining an artery to a vein under the patient's skin, usually in the forearm or wrist, to allow for arterial blood flow directly into the vein. Fistulas should be placed several months prior to the initiation of hemodialysis to allow for proper healing before use. Two to three months after the fistula is surgically formed, the fistula matures creating a larger blood vessel and easier, less painful vascular access. The subsequent increase in flow of arterial blood into the vein permits percutaneous puncture of the blood vessel, allowing needles to be inserted and removed during each hemodialysis treatment. Between hemodialysis treatments, only a small scar and swelling are visible on the patient.

Although fistulas can last for years, there is a risk of infection and stenosis or narrowing of the fistula. Once the fistula becomes occluded, vascular access may be lost requiring placement of either a fistula or a graft in another location. Clot-busting drugs may be used to reverse stenosis of the fistula, however, these medications can cause complications including bleeding disorders, severe allergic reactions and death. When a fistula fails, or the patient's blood vessels are too small to create and maintain a fistula, AV grafts may be used for vascular access.

AV grafts are a reasonable alternative to fistulas, but grafts are not without problems. Grafts are formed by using either an artificial blood vessel or a larger vessel from the patient's own body to internally join an artery and a vein under the patient's skin, usually in the forearm or thigh. The graft is surgically placed close to the surface of the skin and may be utilized within two to four weeks after placement and provide for easier, less painful vascular access.

Grafts, as compared to fistulas, require shorter times to heal before they can be used, but tend to have problems associated with them. Grafts usually do not last as long as fistulas and grafts have greater incidence of stenosis and thrombosis than fistulas. Because grafts are usually artificial and not a vessel obtained from the patient, infection, thrombosis, pseudoaneurysm, hematoma, and stenosis or narrowing of the graft may occur. If any of these complications do arise, vascular access may be lost. To prevent loss of vacular access, the graft must somehow be cleared. Currently, either clot-busting drugs or surgery are the only treatments available. However, these treatments can be very invasive and do not come without risks including bleeding, allergic reactions, pulmonary embolism, cardiac arrest and death.

Catheters provide an access made by means of a narrow plastic tube which is inserted into a large vein, usually in the patient's neck. Catheters are most often used as “bridge” devices, used to bridge the time between the commencement of dialysis treatments (often an emergency) to when the patient's AV fistula or AV graft has matured and is ready for use. Catheters are generally not used as long-term devices as they tend to have higher rates of infection and thrombosis.

If the patient's access is a fistula or graft, the patient's nurse or technician will place two needles into the access at the beginning of each hemodialysis treatment. These needles are connected to dialysis lines (soft plastic tubes) that connect to the hemodialyser. Blood goes to the hemodialyser through one of the dialysis lines, gets cleaned in the hemodialyser, and returns to the patient through the other dialysis lines. If the patient's access is a catheter, the dialysis lines can be connected directly to the catheter without the use of needles.

A fistula is considered the first choice for the patient's access because a fistula generally lasts longer and has the lowest rate of complications such as infections and clotting. However, some patients may not be able to receive a fistula because their blood vessels are not strong enough. A graft is then considered the second choice for the patient's access. Catheters are generally used as a temporary access, but sometimes catheters may provide permanent access. It is possible to switch to a fistula from another type of access.

Whether the access is a fistula, graft or catheter, the patient should care for the access so problems do not develop. The most common problems associated with vascular access include stenosis (narrowing of blood vessel/graft), thrombosis (clotting), and infection.

Venous stenosis is the narrowing of the blood vessel or graft. Physiologically, venous stenosis increases resistance to blood flow, which in turn results in increased venous pressure, decreased blood flow and, ultimately, thrombosis. Moreover, the presence of venous stenosis reduces the efficiency of the hemodialysis treatment. Stenosis can and should be detected prospectively to allow swift, successful treatment. Correction of venous stenoses of greater than fifty percent lumen diameter can result in a significant decrease in the rate of fistula thrombosis and an improvement in access patency. Currently, stenosis is diagnosed by measuring the venous pressure at constant blood flow (200 ml/min) through the hemodialyser. Venous stenosis increases the risk of thrombosis.

Thrombosis is an obstruction of a blood vessel by a clot of coagulated blood formed at the site of obstruction. A thrombus is an aggregation of blood factors, primarily platelets and fibrin with entrapment of cellular elements, frequently causing vascular obstruction at the point of its formation. A thrombus is distinguished from an embolism, in that the embolism is produced by a clot or foreign body brought from a distance. Thromobis results in an elevation of resistance and impairment of access flow. Treatment of access thrombosis requires invasive, time-consuming, and expensive procedures.

Therapeutic interventions for hemodynamically significant stenoses reduce the rate of thrombosis and graft loss and prolong the average use-life of the access. Long-term patency of the access is improved if stenoses are treated prior to thrombus formation as opposed to undertaking angioplasty or surgical revision (with their respective needs for thrombolysis or thrombectomy) after thrombus occlusion of the access has occurred.

Venous stenosis and thrombotic episodes cause the vast majority of access failures in pateints. Additionally, infection or other complications can also result in access failure. The complications of vascular access are not only a major cause of morbidity in hemodialysis patients, but a major cost for the end-stage renal disease treatment program. Access salvage includes prospective monitoring and treatment of outflow stenosis. The direct intra-access measure of blood flow by ultrasound dilution and duplex color flow Doppler technique is the ideal method for detecting venous outflow stenosis. However, conventional and digital subtraction angiography has an advantage in that the total vascular system and blood flow may be visualized. The various treatment modalities for outflow stenosis include use of percutaneous transluminal angioplasty, stents, and surgical correction. The dissolution or destruction of thrombus can be done by surgical, medical and mechanical thrombosis. The various methods being used to prevent graft stenosis include use of dipyridamole and radiation.

All current treatments of stenosis and thrombosis to preserve vascular access are invasive, expensive, and subject the patient to minor and/or severe complications as discussed above. Therefore, there is a continuing need for further developments in the treatment of stenosis and thrombosis to remove debris from grafts, fistulas, vessels and ports in a patient with minimal invasiveness and minimal risk to the patient. In particular, a vascular introducer used in conjunction with an ultrasonic probe, or similar device, to remove debris from grafts, fistulas, vessels and ports in a patient with minimal invasiveness and minimal risk to the patient would further advance the state of the art.

The present invention provides an apparatus and a method for using a vascular introducer in conjunction with an ultrasonic probe to remove a debris from a graft, fistula, vessel, port, or other vascular access device. The debris to be removed by the present invention is any material causing a blockage, occlusion or stenosis of the vascular access device including, but not limited to, thrombi, hematomas, stents, tissue, deposits, plaque, and psuedoaneurysms. The present invention removes the debris from the vascular access device with minimally invasive techniques as well as with minimal risk to the patient.

The present invention is a vascular introducer for insertion into a vascular access device including an elongated shaft having a distal end and a proximal insertion end; an anchoring mechanism that resides within the elongated shaft when the anchoring mechanism is in a retracted position and extends beyond the proximal insertion end of the elongated shaft and engages an inner surface of the vascular access device when the anchoring mechanism is in an extended position; and an activation mechanism that moves the anchoring mechanism from the retracted position to the extended position.

The present invention is a method of clearing a debris from a vascular access device by placing a vascular introducer into the vascular access device; inserting an ultrasonic probe through the vascular introducer and into the vascular access device; and ablating the debris in the vascular access device using the ultrasonic probe; whereby the vascular introducer need not be removed from the vascular access device while the ultrasonic probe is ablating the debris.

The vascular introducer may also include a rotation mechanism that allows the vascular introducer to change direction within the vascular access device without being removed from the vascular access device.

The present invention provides an inexpensive, easy to use, low profile vascular introducer that can clear debris from the vascular access device when used in conjunction with an ultrasonic probe. The vascular introducer is comfortable to a patient and can be used with the ultrasonic probe or other small instruments. The present invention is a disposable, single use vascular introducer for use on a single patient.

### DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a perspective view of a vascular introducer of the present invention inserted in a vascular access device.

FIG. 2 is a perspective view of a vascular introducer of the present invention showing an anchoring mechanism in a retracted position.

FIG. 3 is an enlarged fragmentary view of an insertion end of a vascular introducer of the present invention showing an anchoring mechanism in a retracted position as in FIG. 2.

FIG. 4 is a perspective view of a vascular introducer of the present invention showing an anchoring mechanism in an extended position.

FIG. 5 is a perspective view of a vascular introducer of the present invention showing movement of the vascular introducer relative to a vascular access device while maintaining contact with an inner surface of the vascular access device.

FIG. 6 is an enlarged fragmentary view of an insertion end of a vascular introducer of the present invention showing an anchoring mechanism.

FIG. 7 is an enlarged fragmentary view of an insertion end of a vascular introducer of the present invention showing an alternative embodiment of an anchoring mechanism that includes three anchors.

FIG. 8 is an enlarged fragmentary view of an insertion end of a vascular introducer of the present invention showing an alternative embodiment of an anchoring mechanism that includes a molly-bolt-like structure.

FIG. 9 is an enlarged fragmentary view of an insertion end of a vascular introducer of the present invention showing an alternative embodiment of an anchoring mechanism that includes an inflatable balloon.

FIG. 10 is an enlarged fragmentary view of an insertion end of a vascular introducer of the present invention showing an alternative embodiment of an anchoring mechanism that includes a plurality of screw-like threads.

FIG. 11 is a perspective view of a vascular introducer of the present invention inserted into the vascular system of a patient instead of the vascular access device.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and sprit of the principles of the present invention.

### DETAILED DESCRIPTION

The following terms and definitions are used herein:

“Vascular introducer” as used herein refers to any object of sufficient thickness, density and rigidity to allow for access to a vascular access device.

“Vascular access device” as used herein refers generally to any graft, fistula, vessel, access port or other device providing access to a vascular system of a patient.

“Debris” as used herein refers to any matter causing a blockage, an occlusion or a steno sis of a vascular access device including, but not limited to, thrombi, hematomas, stents, tissue, deposits, plaque, and psuedoaneurysms.

“Ablate” as used herein refers to removing, clearing, or destroying debris. “Ablation” as used herein refers to the removal, clearance, destruction, or taking away of debris.

“Ultrasonic probe” as used herein refers to any medical device utilizing ultrasonic energy with the ability to ablate debris including, but not limited to, probes, elongated wires, and similar devices known to those skilled in the art. The ultrasonic energy of the ultrasonic probe may be in either a longitudinal mode or a transverse mode.

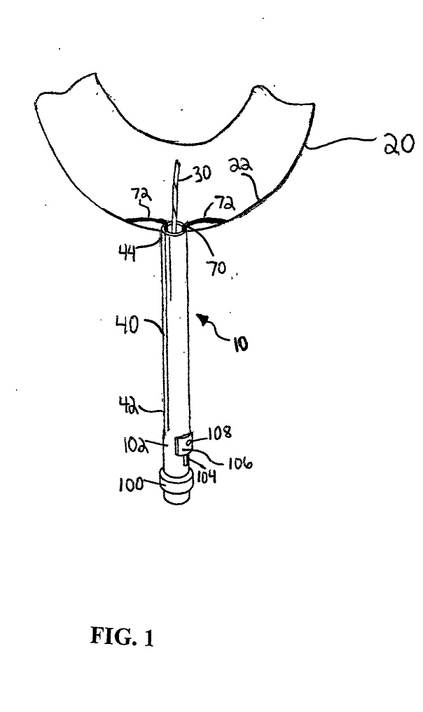

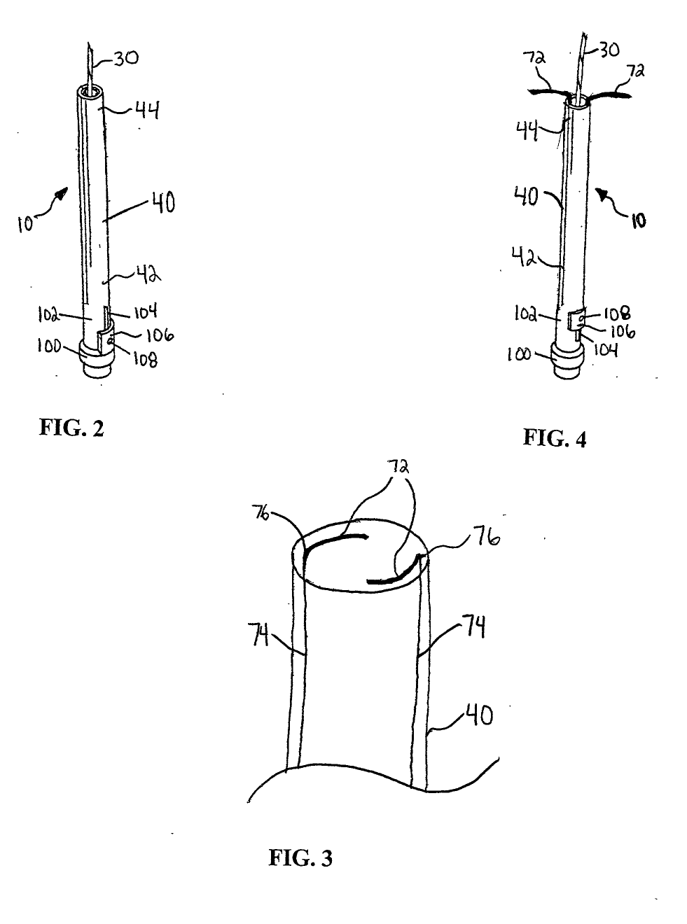

A vascular introducer of the present invention is illustrated generally at 10 in FIG. 1. The vascular introducer 10 can be inserted into a vascular access device 20. The vascular introducer 10 includes an elongated shaft 40, an anchoring mechanism 70, and an activation mechanism 100. The elongated shaft 40 is hollow and has a distal end 42 and a proximal insertion end 44. The elongated shaft 40 houses the anchoring mechanism 70 and allows the anchoring mechanism 70 to move from a retracted position (shown in FIG. 2 and FIG. 3) to an extended position (shown in FIG. 1 and FIG. 4) by the activation mechanism 100. In the extended position shown in FIG. 1 and FIG. 4, the anchoring mechanism 70 extends beyond the proximal insertion end 44 of the elongated shaft 40 and engages an inner surface 22 of the vascular access device 20, maintaining contact between the vascular introducer 10 and the vascular access device 20. In the retracted position shown in FIG. 2 and FIG. 3, the anchoring mechanism 70 resides within the elongated shaft 40 and does not extend beyond the proximal insertion end 44 of the elongated shaft 40. An ultrasonic probe 30 can be inserted through the vascular introducer 10 into the vascular access device 20 for ablating (with ultrasonic energy emitted from the ultrasonic probe 30) any debris causing a blockage, occlusion or stenosis of the vascular access device 20. In an alternative embodiment of the present invention, a rotation mechanism 90 permits the vascular introducer 10 and the ultrasonic probe 30 inside the vascular introducer 10 to change direction and therefore change the area of ablation without being removed from the vascular access device 20.

By utilizing the vascular introducer 10 of the present invention with an ultrasonic probe 30, it is possible to remove debris including, but not limited to, thrombi, hematomas, stents, tissue, deposits, plaque, and psuedoaneurysms from the vascular access device 20 with minimal risk to a patient while maintaining minimum invasiveness. While the vascular introducer 10 of the present invention can be used with any ultrasonic probe 30, it is appreciated by those skilled in the art that the vascular introducer 10 of the present invention can also be used with other medical devices and has applications beyond ultrasonic probes. In a preferred embodiment of the present invention, the vascular introducer 10 is used with an ultrasonic probe 30 operating in a transverse mode. Transversely vibrating ultrasonic probes for tissue ablation are described in the Assignee's co-pending patent applications (U.S. Ser. No. 09/766,015, U.S. Serial No. 60/178,901 and U.S. Serial No. 60/225,060) which further describe the design parameters for such a probe and its use in ultrasonic devices for ablation, and the entirety of these applications are hereby incorporated by reference.

The anchoring mechanism 70 of the vascular introducer 10 prevents removal or detachment of the vascular introducer 10 from the vascular access device 20. Many types of anchoring mechanisms 70 are commonly used with other medical devices and are well known to those of skill in the art. The anchoring mechanisms 70 that are encompassed within the scope of the present invention include, but are not limited to, a plurality of wing-like objects, a plurality of molly-bolt-like structures, an inflatable balloon, a plurality of screw-like threads, and similar structures.

As best shown in FIG. 3, the anchoring mechanism 70 of the vascular introducer 10 includes at least one anchor 72 and at least one anchor extension rod 74 that is connected to the anchor 72 by a connecting means 76. The present invention discloses one to a plurality of anchors 72 with a separate anchor extension rod 74 associated with each anchor 72. In a preferred embodiment of the present invention (as shown in FIG. 1 and FIG. 3), the anchoring mechanism 70 includes two anchors 72 and the associated anchor extension rods 74. However, alternative embodiments of the present invention disclose the anchoring mechanism 70 including one, three, four, five, or more anchors 72 and the associated anchor extension rods 74. FIG. 7 shows an alternative embodiment of the present invention wherein the anchoring mechanism 70 includes three anchors 72. The connecting means 76 can be any means of connecting the anchor 72 to the anchor extension rod 74 known in the art, including, but not limited to, adhesives, welding, coupling, clamping, fastening, and the like. In an alternative embodiment of the present invention, the anchoring mechanism 70 can be a single, continuous piece that includes the anchor 72 and the anchor extension rod 74, making the connecting means 76 unnecessary in this embodiment.

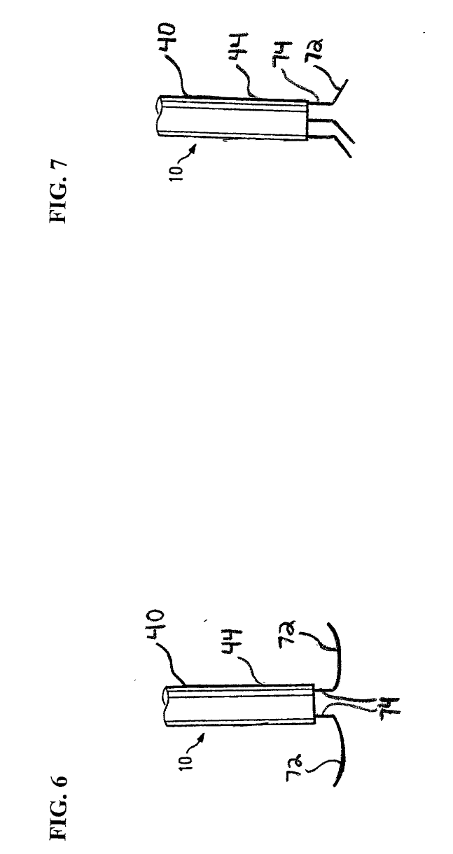

As best shown in FIG. 1 and FIG. 4, a preferred embodiment of the present invention features the anchoring mechanism 70 including the anchor 72 that is a wing-like object that flexes outward from the proximal insertion end 44 of the elongated shaft 40 of the vascular introducer 10 after the vascular introducer is placed in the vascular access device 20. The anchor 72 that is a wing-like object can have any shape including, but not limited to, a curved shape (FIG. 6) or a straight shape (FIG. 7). The anchoring mechanism 70 is movable between a retracted position (shown in FIG. 2 and FIG. 3) and an extended position (shown in FIG. 1 and FIG. 4). In the retracted position (shown in FIG. 2 and FIG. 3), the elongated shaft 40 houses the anchoring mechanism 70 including the anchor 72 and the anchor extension rod 74. As best shown in FIG. 3, in the retracted position, the anchor 72 is located inside the elongated shaft 40 and does not extend beyond the proximal insertion end 44 of the elongated shaft 40. The retracted position of the anchoring mechanism 70 is used to insert and remove the vascular introducer 10 from the vascular access device 20. As best shown in FIG. 1, in the extended position, the anchor 72 extends beyond the proximal insertion end 44 of the elongated shaft 40 and the anchor 72 rotates to engage the inner surface 22 of the vascular access device 20, maintaining contact between the vascular introducer 10 and the vascular access device 20. The extended position of the anchoring mechanism 70 is used to prevent removal or detachment of the vascular introducer 10 from the vascular access device 20 during the treatment procedure with the ultrasonic probe 30. In a preferred embodiment of the present invention, the anchor mechanism 70 is such that the vascular introducer 10 maintains a low profile once inside the vascular access device 20 and prevents removal of the vascular introducer 10 once the vascular introducer 10 is placed in the vascular access device 20.

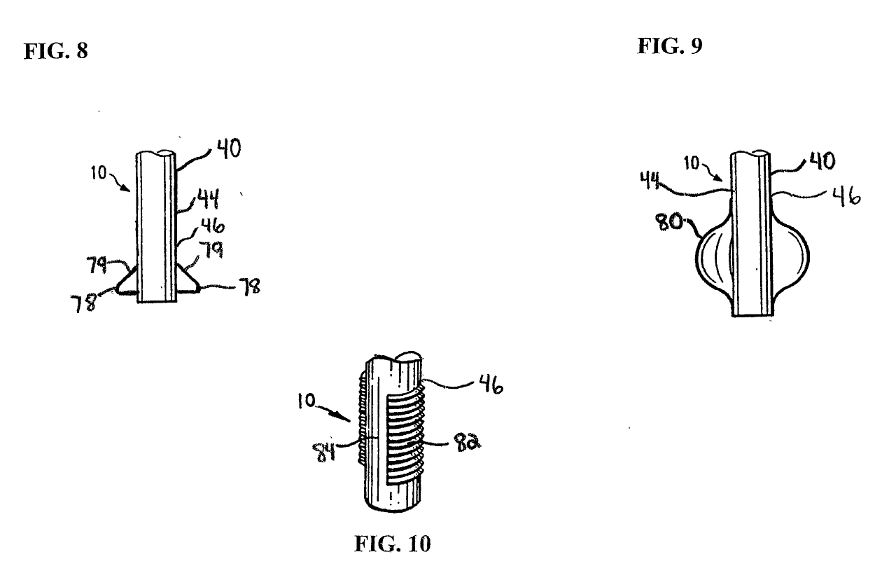

FIG. 8 shows an alternative embodiment of the present invention wherein the anchor mechanism 70 includes one to a plurality of molly-bolt-like structures 78 on an external surface 46 of the proximal insertion end 44 of the elongated shaft 40. The molly-bolt-like structure 78 has a flange 79 that opens out and grips the inner surface 22 of the vascular access device 20, making it harder for the vascular introducer 10 to be pulled out of the vascular access device 20. The molly-bolt-like structures 78 prevent removal or detachment of the vascular introducer 10 once the vascular introducer 10 is placed in the vascular access device 20.

FIG. 9 shows an alternative embodiment of the present invention wherein the anchor mechanism 70 includes an inflatable balloon 80 mounted on the external surface 46 of the proximal insertion end 44 of the elongated shaft 40. The inflatable balloon 80 can be inflated to an expanded condition to secure the vascular introducer 10 in the vascular access device 20.

FIG. 10 shows an alternative embodiment of the present invention wherein the anchor mechanism 70 includes one to a plurality of screw-like threads 82 on the external surface 46 of the proximal insertion end 44 of the elongated shaft 40. The screw-like threads 82 are discontinuous and define at least one unthreaded groove 84. The screw-like threads 82 prevent removal or detachment of the vascular introducer 10 once the vascular introducer 10 is placed in the vascular access device 20. In another alternative embodiment of the present invention, the screw-like threads 82 are continuous around the external surface 46 of the proximal insertion end 44 of the elongated shaft 40 and the unthreaded groove 84 is not present in this embodiment.

In an alternative embodiment of the present invention, the anchoring mechanism 70 of the vascular introducer 10 includes a coaxial sleeve that is slidably disposed within the elongated shaft 40. An outside diameter of the coaxial sleeve is slightly less than an inside diameter of the elongated shaft 40, so that there is a coaxial clearance space between the coaxial sleeve and the elongated shaft 40. The anchoring mechanism 70 is attached to and extends from a proximal end of the coaxial sleeve. The distal end of the coaxial sleeve engages the activation mechanism 100 in a manner similar to that as will be discussed below. Thus, the anchoring mechanism 70 can moved from the retracted position to the extended position by the activation mechanism 100 in a manner similar to that discussed above. In this multi-lumen embodiment of the present invention, the vascular introducer 10 includes at least two shafts or sleeves.

As shown in FIGS. 1, 2, 4, and 5, the activation mechanism 100 includes a hollow, tubular central portion 102, an axial slot 104, a button 106, and a stem 108. The axial slot 104 communicates with the interior of the central portion 102. An outer end of the stem 108 is attached the button 106 so that the stem 108 can travel in the axial slot 104. The inner end of the stem 108 engages the distal end of the anchor extension rod 74. Movement of the button 106 causes the stem 108 to slidably travel in the axial slot 104 and causes the anchoring mechanism 70 to move axially within and with respect to the elongated shaft 40, as explained below.

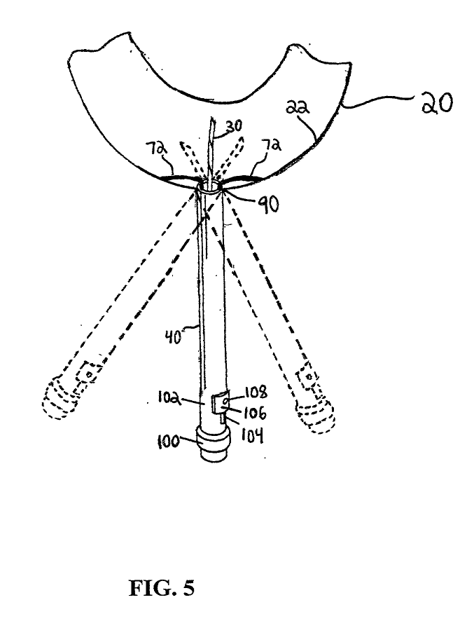

When the button 106 is a first lower position (FIG. 2) the anchoring mechanism 70 is in the retracted position (FIG. 2 and FIG. 3). The retracted position of the anchoring mechanism 70 is used to insert and remove the vascular introducer 10 from the vascular access device 20. After the vascular introducer 10 is placed in the vascular access device 20, movement of the button 106 to a second upper position (FIG. 4) moves the anchoring mechanism 70 to the extended position (FIG. 1 and FIG. 4) where the anchor 72 extends beyond the proximal insertion end 44 of the elongated shaft 40 and the anchor 72 rotates to engage the inner surface 22 of the vascular access device 20, maintaining contact between the vascular introducer 10 and the vascular access device 20. The extended position of the anchoring mechanism 70 is used to prevent removal or detachment of the vascular introducer 10 from the vascular access device 20 during the treatment procedure with the ultrasonic probe 30. As shown in FIG. 5, in the extended position, the anchoring mechanism 70 allows the vascular introducer 10 to be adjusted into many different positions as required while preventing removal of the vascular introducer 10 from the vascular access device 20.

In an alternative embodiment of the present invention, the activation mechanism 100 includes a “push-button” mechanism to extend the anchoring mechanism 70 from the retracted position (FIG. 2 and FIG. 3) to the extended position (FIG. 1 and FIG. 4). The push-button mechanism is similar to that commonly found in a ball-point pen and known to those skilled in the art. When the push-button is pressed, the push-button pushes forward a thrust tube together with the anchoring mechanism 70. When the push-button is pressed, the catches of the thrust tube are fully inserted into the fixed slots in the elongated shaft 40. When the push-button is released, the action of a large spring retracts the anchoring mechanism 70. The catches of the thrust tube (connected to the anchoring mechanism 70) engage with the small teeth on a rotating sleeve. The anchoring mechanism 70 is then in the extended position. When the push-button is pressed again, the catches of the thrust tube plunge into the fixed slots. The rotating sleeve, which is spring-loaded by a small spring, bears on the sharp edges of the fixed slots, while at the same time the rotating sleeve rotates an amount corresponding to one tooth. When the thrust tube moves back, the rotating sleeve is first lifted and, at the same time, turned. The catches of the thrust tube can then plunge into the large tooth gaps of the rotating sleeve, so that the anchoring mechanism 70 returns to the retracted position inside the elongated shaft 40. The action is controlled by the rotating sleeve, which performs a small rotational movement whenever the push-button is actuated. Those skilled in the art will appreciate that any type of push-button mechanism known in the art would be applicable to the present invention.

In an alternative embodiment of the present invention, the activation mechanism 100 includes a “ball catch” that functions as a retraction mechanism. When the push button is pressed, a ball rotates in a clockwise direction in a cam recess in a side of a cylindrical sleeve attached to the push-button. The position of the ball within the cam recess determines the position of the anchoring mechanism 70. When the push-button is pressed, the ball is at the top holding point of the cam recess and the ball is held there by the pressure of a spring. The anchoring mechanism 70 is then in the extended position. When the push-button is pressed again, the ball goes to the bottom holding point of the cam recess, and the anchoring mechanism 70 returns to the retracted position inside the elongated shaft 40. Those skilled in the art will appreciate that any type of ball catch mechanism known in the art would be applicable to the present invention.

In an embodiment of the present invention, the vascular introducer 10 includes the rotation mechanism 90 to allow for a reversal of direction. Referring to FIG. 5, the vascular introducer 10 is shown in solid lines and broken lines at two different angular positions with respect to the vascular access device 20. In FIG. 5, it is understood that the vascular introducer 10 may be moved into any intermediate position between (and even beyond) these different angular positions when desired. The rotation mechanism 90 allows the vascular introducer 10 to be rocked into various relationships relative to the vascular access device 20 while the anchoring mechanism 70 maintains contact with the inner surface 22 of the vascular access device 20. The rotation mechanism 90 allows the ultrasonic probe 30 to change direction and therefore the area of ablation of the ultrasonic probe 30 without being removed from the vascular introducer 10 and therefore the vascular access device 20. In a preferred embodiment of the present invention, the rotation mechanism 90 can be provided by a flexible portion on the external surface 46 of the elongated shaft 40 that allows the vascular introducer 10 to bend in an alternate directions. In an alternative embodiment of the present invention, the rotation mechanism 90 can be provided by a ball-and-socket mechanism. The ball-and-socket mechanism is a mechanical connection that allows some relative angular motion in nearly all directions. The ball-and-socket mechanism includes a member with a spherical end placed within a socket recessed to fit it, thus permitting relative movement in nearly all directions within a given cone, or a cutout in the socket. In another embodiment of the present invention, the rotation mechanism 90 can be provided by a hinged mechanism.

It is noted, that the vascular introducer 10 can also be moved axially through the interior of the vascular access device 20 as required by the treatment procedure. However, when the anchoring mechanism 70 is in the extended position (FIG. 1 and FIG. 4), it is difficult to remove the vascular introducer 10 from the vascular access device 20 because the anchoring mechanism 70 maintains contact with the inner surface 22 of the vascular access device 20 preventing removal of the vascular introducer 10 from the vascular access device 20.

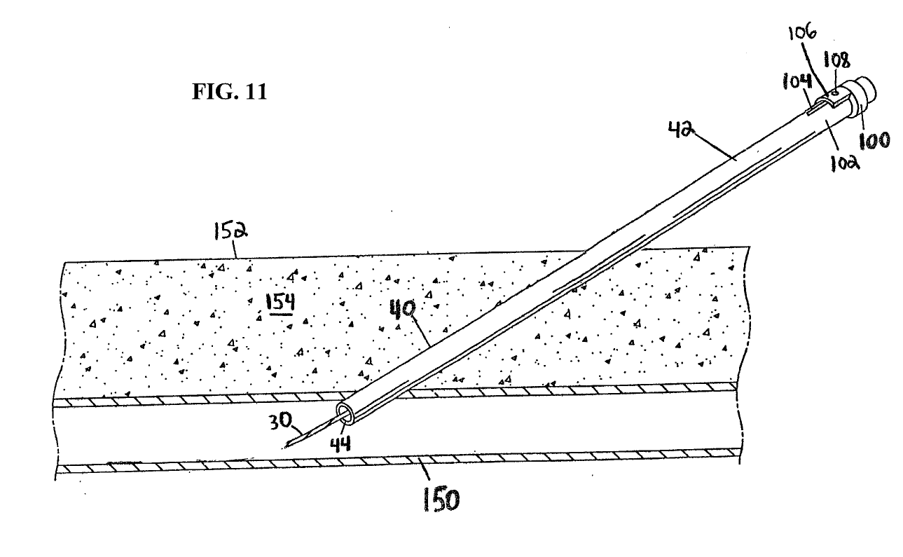

The vascular introducer 10 can have varying lengths and diameters. The length and diameter of the vascular introducer 10 used in a particular treatment procedure will depend on the type of the ultrasonic probe 30 selected and the extent and length to which the ultrasonic probe 30 will be inserted into the vascular access device 20. FIG. 1 shows the vascular introducer 10 with a shorter length because the vascular introducer 10 is inserted into the vascular access device 20. FIG. 11 shows the vascular introducer 10 with a longer length because the vascular introducer 10 is inserted into a vascular system of a patient instead of the vascular access device 20 of FIG. 1. Thus, the vascular introducer 10 of FIG. 11 requires a longer length than the vascular introducer 10 of FIG. 1. The inside diameter of the elongated shaft 40 of the vascular introducer 10 is large enough to allow the ultrasonic probe 30 to be freely passed through it. For any treatment procedure, the vascular introducer 10 must be of a sufficient outer diameter to facilitate insertion of the vascular introducer 10 into the vascular access device 20, and of a sufficient inner diameter to enable acceptance of the ultrasonic probe 30. The exact length and diameter of the vascular introducer 10 will be determined by the requirements of the treatment procedure and the specific patient requirements.

In a preferred embodiment of the present invention, the vascular introducer 10 has a diameter that is approximately the same over their entire length, that is, the distal end 42 and the proximal insertion end 44 of the vascular introducer 10 are approximately uniform in diameter. The uniform diameter of the vascular introducer 10 allows catheters and guides to be introduced into the vascular introducer 10 in addition to the ultrasonic probe 30, and permits the use of the vascular introducer 10 in standard-configuration endovascular procedures. In a preferred embodiment of the present invention, the diameter of the vascular introducer 10 is less than or equal to 9 french. In an alternative embodiment of the present invention (not shown in the drawings), the distal end 42 of the vascular introducer 10 has a diameter that is larger than the diameter of the proximal insertion end 44 of the vascular introducer 10. The larger diameter of the distal end 42 of the vascular introducer 10 allows the vascular introducer 10 to be used in conjunction with ultrasonic probes, catheters and guides that do not have a uniform shape (i.e., non-cylindrical) or have “bulk” on the distal end.

The vascular introducer 10 of the present invention can be made of any material having sufficient strength, thickness, density and rigidity to allow for the introduction of the vascular introducer 10 through a surface of the vascular access device 20. Additionally, the material that the vascular introducer 10 is made of should maintain the structural integrity and flexibility once the ultrasonic probe 30 is inserted. The vascular introducer 10 should also be composed of a material that is capable of being sterilized by, for example, gamma irradiation or ethylene oxide gas (ETO), without losing its structural integrity. Such materials include, but are not limited to, substantially rigid non-metallic materials or plastic materials such as polytetrafluoroethylene (PTFE), polyethylene, polypropylene, polyimide, silicone, polyetherimide, or other plastics that those skilled in the art know are commonly used in medical devices. Ceramic materials can also be used, and have the added benefit of being able to be sterilized by heat and pressure, such as in an autoclave. In a preferred embodiment of the present invention, polytetrafluoroethylene (PTFE) is used to fabricate a strong, flexible, disposable vascular introducer 10 that is easily sterilized by irradiation or ethylene oxide gas (ETO). In an alternative embodiment of the present invention, the vascular introducer 10 can be made of combinations of the aforementioned materials depending on the requirements of the treatment procedure. The vascular introducer 10 may employ two or more materials to give the desired combination of strength and flexibility. For example, the vascular introducer 10 may include a rigid ceramic distal end 42 and a more flexible plastic proximal insertion end 44, capable of flexing with the ultrasonic probe 30.

The outer shape of the vascular introducer 10 is designed to be atraumatic and allows easy placement of the vascular introducer 10 within the vascular access device 20. The anchoring mechanism 70 effectively holds the vascular introducer 10 in place once the vascular introducer 10 is positioned within the vascular access device 20. An important feature of the vascular introducer 10 is that lateral or rocking motion of the vascular introducer 10 does not cause removal of the vascular introducer 10 from the vascular access device 20 because the anchoring mechanism 70 effectively holds the vascular introducer 10 in place and maintains contact with the inner surface 22 of the vascular access device 20 during use.

FIG. 11 shows an alternative embodiment of the present invention wherein the vascular introducer 10 is inserted into the vascular system of the patient instead of the vascular access device 20 of FIG. 1. In FIG. 11, the vascular introducer 10 is shown inserted into a blood vessel 150 through a skin 152 and a subcutaneous tissue 154 of the patient. While FIG. 11 shows the vascular introducer 10 inserted into a blood vessel 150, the vascular introducer 10 may similarly be inserted into any body lumen as will be readily appreciated to those skilled in the art. In FIG. 11, the ultrasonic probe 30 is shown extending from the proximal insertion end 44 of the elongated shaft 40 into the blood vessel 150 for ablation of the debris.

The vascular introducer 10 is comfortable to a patient and can be used with small instruments, other than those discussed above, that are know to those skilled in the art. An additional advantage of the vascular introducer 10 of the present invention is that it is relatively inexpensive to manufacture. Thus, the vascular introducer 10 may be disposed of after a single use. In a further embodiment of the present invention, the vascular introducer 10 is meant for a single use only.

The vascular introducer 10 of the present invention can be used to remove debris including, but not limited to, thrombi, hematomas, stents, tissue, deposits, plaque, and psuedoaneurysms from the vascular access device 20. A significant advantage of the present invention over the prior art is that the anchoring mechanism 70 and the rotation mechanism 90 permit the vascular introducer 10 and the ultrasonic probe 30 inside the vascular introducer 10 to change direction and therefore change the area of ablation of the ultrasonic probe 30 without being removed from the vascular access device 20.

The method of clearing debris from the vascular access device 20 of the present invention includes placing the vascular introducer 10 into the vascular access device 20; inserting the ultrasonic probe 30 into the vascular access device 20 through the vascular introducer 10; and ablating any debris in the vascular access device 20 using the ultrasonic energy emitted from the ultrasonic probe 30 whereby the ultrasonic probe 30 need not be removed from the vascular introducer 10 and the vascular access device 20 during the ablation process. The method of the present invention can also include rotating the vascular introducer 10 and the ultrasonic probe 30 inside the vascular introducer 10 to change the direction and therefore change the area of ablation of the ultrasonic probe 30 without removing the vascular introducer 10 from the vascular access device 20. In a preferred embodiment of the present invention, the placement of the vascular introducer 10 can be achieved by an apical puncture of the vascular access device 20. In a preferred embodiment of the present invention, the ultrasonic probe 30 functions in a transverse mode. In a preferred embodiment of the present invention, the ultrasonic probe 30 is sufficiently flexible to prevent puncture through the side or back of the vascular access device 20.

Once the debris has been destroyed, the user of the vascular introducer 10 (e.g., a physician or a medical technician) can rotate the vascular introducer 10 to the next site having debris and needing treatment, and repeat the process. It is anticipated that in an embodiment of the method of treatment of the present invention, more than one treatment may be required to clear all debris from the vascular access device 20.

Providing ultrasonic energy to the ultrasonic probe 30 generates an area of ablation along the longitudinal axis of the ultrasonic probe 30. By bringing the area of ablation of the ultrasonic probe 30 near the debris in the vascular access device 20, the debris is destroyed and the blockage, occlusion or stenosis is cleared. Sweeping the ultrasonic probe 30 over the debris creates a tissue-destructive effect within the vicinity of the ultrasonic probe 30. The sweeping of the ultrasonic probe 30 over the debris is preferably in a windshield-wiper fashion with the debris removed in all areas adjacent to the area of ablation of the ultrasonic probe 30. Alternatively, the sweeping of the ultrasonic probe 30 may be in a longitudinal, spiral, or any other fashion necessary to destroy the debris.

The apparatus and method for using the vascular introducer 10 with the ultrasonic probe 30 of the present invention discloses an inexpensive, easy to use, low profile vascular introducer 10 that can clear blockages, occlusions or stenosis of the vascular access device 20 when used in conjunction with an ultrasonic probe 30. A significant advantage of the present invention over the prior art is that the anchoring mechanism 70 and the rotation mechanism 90 permit the vascular introducer 10 and the ultrasonic probe 30 inside the vascular introducer 10 to change direction and therefore change the area of ablation of the ultrasonic probe 30 without being removed from the vascular access device 20.

Variations, modifications, and other implementations of what is described herein will occur to those of ordinary skill in the art without departing from the spirit and scope of the present invention as claimed. Accordingly, the present invention is to be defined not by the preceding illustrative description but instead by the spirit and scope of the following claims.

What is claimed is:

1. A vascular introducer for insertion into a vascular access device comprising:
an elongated shaft having a distal end and a proximal insertion end;
an anchoring mechanism that resides within the elongated shaft when the anchoring mechanism is in a retracted position and extends beyond the proximal insertion end of the elongated shaft and engages an inner surface of the vascular access device when the anchoring mechanism is in an extended position; and
an activation mechanism that moves the anchoring mechanism from the retracted position to the extended position.

2. The vascular introducer of claim 1, wherein the vascular access device is a graft, a fistula, a vessel or an access port.

3. The vascular introducer of claim 1, wherein the extended position of the anchoring mechanism maintains contact between the vascular introducer and the vascular access device to prevent removal or detachment of the vascular introducer from the vascular access device.

4. The vascular introducer of claim 1, further comprising a rotation mechanism that allows the vascular introducer to change direction within the vascular access device without being removed from the vascular access device.

5. The vascular introducer of claim 1, wherein an ultrasonic probe can be inserted through the vascular introducer into the vascular access device for ablation of a debris in the vascular access device.

6. The vascular introducer of claim 5, wherein the debris is any material causing a blockage, an occlusion or a stenosis of the vascular access device.

7. The vascular introducer of claim 5, wherein the ultrasonic probe ablates the debris by emitting ultrasonic energy.

8. The vascular introducer of claim 1, wherein the vascular introducer is for a single use on a single patient.

9. A vascular introducer for access to a vascular system of a mammal through a vascular access device comprising:
an elongated shaft having a wall of sufficient strength to penetrate through a surface of the vascular access device;
at least one anchor that maintains contact between the vascular introducer and the vascular access device to prevent removal or detachment of the vascular introducer from the vascular access device; and
a rotation means that allows the vascular introducer to change direction within the vascular access device without being removed from the vascular access device.

10. The vascular introducer of claim 9, wherein the vascular access device is a graft, a fistula, a vessel or an access port.

11. The vascular introducer of claim 9, wherein at least one anchor is movable between a retracted position where at least one anchor resides within the elongated shaft and an extended position where at least one anchor extends beyond a proximal insertion end of the elongated shaft and engages an inner surface of the vascular access device.

12. The vascular introducer of claim 11, further comprising an activation mechanism that moves at least one anchor from the retracted position to the extended position.

13. The vascular introducer of claim 9, wherein an ultrasonic probe can be inserted through the vascular introducer into the vascular access device for ablation of a debris in the vascular access device.

14. The vascular introducer of claim 13, wherein the debris is any material causing a blockage, an occlusion or a stenosis of the vascular access device.

15. The vascular introducer of claim 13, wherein the ultrasonic probe ablates the debris by emitting ultrasonic energy.

16. The vascular introducer of claim 9, wherein the vascular introducer is for a single use on a single patient.

17. A method of clearing a debris from a vascular access device comprising:
placing a vascular introducer into the vascular access device;
inserting an ultrasonic probe through the vascular introducer and into the vascular access device; and
ablating the debris in the vascular access device using the ultrasonic probe;
whereby the vascular introducer need not be removed from the vascular access device while the ultrasonic probe is ablating the debris.

18. The method of claim 17, wherein the vascular access device is a graft, a fistula, a vessel or an access port.

19. The method of claim 17, wherein the debris is any material causing a blockage, an occlusion or a stenosis of the vascular access device.

20. The method of claim 17, wherein the ultrasonic probe ablates the debris by emitting ultrasonic energy.

21. The method of claim 17, further comprising rotating the vascular introducer to change direction and an area of ablation of the ultrasonic probe within the vascular access device without removing the vascular introducer from the vascular access device.

22. The method of claim 17, wherein at least one anchor maintains contact between the vascular introducer and the vascular access device to prevent removal or detachment of the vascular introducer from the vascular access device.

23. The method of claim 17, wherein at least one anchor is movable between a retracted position where at least one anchor resides within the vascular introducer and an extended position where at least one anchor extends beyond a proximal insertion end of the vascular introducer and engages an inner surface of the vascular access device.

24. The method of claim 23, wherein an activation mechanism moves at least one anchor from the retracted position to the extended position.

25. The method of claim 17, wherein the vascular introducer is for a single use on a single patient.

26. A method of using a vascular introducer with an ultrasonic probe to remove a debris from a graft, a fistula, a vessel, a port, or other device providing vascular access comprising:
positioning a proximal insertion end of the vascular introducer into the device providing vascular access;
inserting the ultrasonic probe through the vascular introducer and into the device providing vascular access;
removing the debris in the device providing vascular access using an ultrasonic energy emitted from the ultrasonic probe; and
rotating the vascular introducer within the device providing vascular access to change a direction of the ultrasonic energy emitted from the ultrasonic probe without removing the vascular introducer from the device providing vascular access.

27. The method of claim 26, wherein the debris is any material causing a blockage, an occlusion or a stenosis of the device providing vascular access.

28. The method of claim 26, wherein at least one anchor maintains contact between the vascular introducer and the device providing vascular access to prevent removal or detachment of the vascular introducer from the device providing vascular access.

29. The method of claim 26, wherein at least one anchor is movable between a retracted position where at least one anchor resides within the vascular introducer and an extended position where at least one anchor extends beyond the proximal insertion end of the vascular introducer and engages an inner surface of the device providing vascular access.

30. The method of claim 29, wherein an activation mechanism moves at least one anchor from the retracted position to the extended position.

31. The method of claim 26, wherein the vascular introducer is for a single use on a single patient.
